(19) United States
(12) Patent Application Publication
(10) Pub. No.: US 2019/0015491 A1
(43) Pub. Date: Jan. 17, 2019

(54) NEOEPITOPE RNA CANCER VACCINE

(71) Applicant: VACCIBODY AS, Oslo (NO)

(72) Inventor: Agnete Brunsvik Fredriksen, Rælingen (NO)

(73) Assignee: VACCIBODY AS, Oslo (NO)

(21) Appl. No.: 16/068,441

(22) PCT Filed: Jan. 5, 2017
(86) PCT No.: PCT/EP2017/050216

§ 371 Date: Jul. 6, 2018

(30) Foreign Application Priority Data
Jan. 8, 2016 (EP) 16150574.8
Jul. 7, 2016 (EP) 16178393.1
Jul. 11, 2016 (EP) 16178816.1

(51) Int. Cl. A61K 39/00; A61P 35/00; C12N 15/11; C12N 15/66

(52) U.S. Cl. CPC A61K 39/0011; A61P 35/00; A61K 2039/53; C12N 15/66; C12N 15/11

(57) ABSTRACT

The present invention relates to an anticancer vaccine comprising polynucleotides or polypeptides, methods of treatment of cancer wherein such an anticancer vaccine is used as well as methods for producing the vaccine. The vaccine comprises a polynucleotide comprising a nucleotide sequence encoding a targeting unit, a dimerization unit, a first linker and an antigenic unit, wherein said antigenic unit comprises n-1 antigenic subunits, each subunit comprising at least a part of a cancer neoepitope sequence and a second linker and said antigenic unit further comprising a final cancer neoepitope sequence, wherein n is an integer of from 3 to 50, or the vaccine comprises a polypeptide encoded by the polynucleotide or a dimeric protein consisting of two polypeptides encoded by the polynucleotide.

### FIELD OF INVENTION

The present invention relates to an anticancer vaccine comprising neoepitope polynucleotides or polypeptides, methods of treatment of cancer wherein such an anticancer vaccine is used as well as methods for producing the vaccine.

### BACKGROUND OF INVENTION

Although treatment of cancer has been improved over the past few decades in particularly due to early detection and diagnosis, which has significantly increased the survival, only about 60% of patients diagnosed with cancer are alive 5 years after the diagnosis.

Most of the cancer treatments in use are surgical procedures, radiation and cytotoxic chemotherapeutics; however they all have serious side effects. Recently also treatment using antibodies directed towards known cancer associated antigens is used.

Within the last few years cancer immune therapies targeting cancer cells with the help of the patient's own immune system, i.e. cancer vaccines, have attracted interest because such therapies may reduce or even eliminate some of the sideeffects seen in the traditional cancer treatment.

The foundation of immunology is based on self-nonself discrimination. Most of pathogens inducing infectious diseases contain molecular signatures that can be recognized by the host and trigger immune responses, however tumor cells are derived from normal cells, and do not generally express any molecular signatures, making them more difficult to be distinguished from normal cells

Nevertheless, most tumor cells express different classes of tumor antigens. One class of tumor antigens are the so-called tumor associated antigens, i.e. antigens expressed in a low level in normal tissues and expressed at a much higher level in tumor tissue. Such tumor-associated antigens have been the target for cancer vaccines for the last decade. However, immunological treatment directed towards tumor associated antigens exhibit several challenges, in that the tumor cells may evade the immune system by downregulating the antigen in question, and the treatment may also lead to toxicities due to normal cell destruction.

Recently, another class of tumor antigens have been identified, the so-called tumor neoantigens or tumor specific-antigens. Tumor neoantigens arise due to one or more mutations in the tumor genome leading to a change in the amino acid sequence of the protein in question. Since these mutations are not present in normal tissue, the sideeffects of the treatment directed towards the tumor associated antigens do not arise with an immunologic treatment towards tumor neoantigens.

The average number of somatic, tumor-specific non-synonymous mutations is for malignant melanoma between 100 and 120. Some of the genetic alterations can be recognized by the immune system, representing ideal antigens. Animal models have confirmed the utility of immunization with tumor neoantigens, and two clinical trials have been initiated, one with a vaccine comprising up to 10 mutated proteins and the other with an RNA vaccine (IVAC MUTANOME). The RNA vaccine comprises 2 RNA molecules each comprising five different mutation-encoding sequences.

However, by administration of either several different proteins or several RNA sequences it is difficult to control the immunological response to the various proteins administered or expressed in vivo.

Accordingly, there is a need for a more efficient vaccine ensuring expression of the mutated proteins either in vivo or in vitro and ensure delivery of the antigen as well as activation of the antigen presenting cells needed to elicit a strong T cell response.

### SUMMARY OF INVENTION

The present invention relates to an anticancer vaccine being directed to a plurality of neoepitopes from tumor neoantigens. Thereby, a personalized neoantigen vaccine is obtained that specifically targets the identified tumor antigens.

In one aspect, the present invention relates to a therapeutic anticancer vaccine comprising an immunologically effective amount of a polynucleotide comprising a nucleotide sequence encoding an antigenic unit comprising
from 2 to 50 antigenic subunits, each subunit comprising at least a part of a cancer neoepitope sequence and a linker
a final cancer neoepitope sequence.

In a preferred embodiment the linker is a flexible linker. It is preferred that the linker is non-immunogenic. In another preferred embodiment the linker is identical in all antigenic subunits.

The linker is preferably a Serine-Glycine linker.

In one embodiment the length of the linker is from 4 to 20 amino acids. In a preferred embodiment the length of the linker is from 10 to 15 amino acids. In a particular embodiment the length of the linker is from 10 amino acids.

In one embodiment of the present invention the vaccine comprises one copy of each cancer neoepitope. In another embodiment the vaccine comprises at least two copies of at least one cancer neoepitope.

In one embodiment the cancer neoepitope sequence has a length of from 7 to 30 amino acids. In a preferred embodiment the cancer neoepitope sequence has a length of from 7 to 10 amino acids. In a particular embodiment the cancer neoepitope sequence has a length of from 13 to 30 amino acids.

In one embodiment each cancer neoepitope sequence has identical length.

In a preferred embodiment the cancer neoepitope is positioned essentially in the middle of the cancer neoepitope sequence. Preferably, the cancer neoepitope sequence is a subsequence of a cancer neoantigen.

It is preferred that the most hydrophobic antigenic subunit(s) is/are substantially the middle of the antigenic unit and the most hydrophilic antigenic subunit(s) is/are at the ends of the antigenic unit.

In one embodiment of the present invention the length of the antigenic unit is from about 100 amino acids to about a 1000 amino acids.

In one embodiment n is an integer between 3 and 30. In a more specific embodiment n is an integer between 10 and 20.

In another aspect, the present invention relates to a polynucleotide comprising the nucleotide sequence as defined herein.

Yet another aspect of the present invention relates to a polypeptide encoded by the polynucleotide as defined herein.

In a further aspect, the present invention relates to use of a vaccine as defined herein for treatment of cancer.

The present invention also relates to a vector comprising the nucleotide sequence as defined herein.

Another aspect of the present invention relates to a host cell comprising a nucleotide sequence encoding the antigenic unit as defined herein or comprising the vector as defined herein.

The present invention also relates to a method for preparing a vaccine comprising an immunologically effective amount of the polypeptide as defined herein, the method comprising
a. transfecting a polynucleotide as defined in claim 21 into a cell population;
b. culturing the cell population;
c. collecting and purifying the polypeptide expressed from the cell population;
d. mixing the polypeptide obtained under step c) with a pharmaceutically acceptable carrier thereby obtaining the vaccine.

In yet another aspect the present invention relates to a method for preparing a vaccine comprising an immunologically effective amount of the polynucleotide as defined herein, said method comprising
a. preparing the polynucleotide as defined herein;
b. mixing the polynucleotide obtained under step a) with a pharmaceutically acceptable carrier, thereby obtaining the vaccine.

In one embodiment the method further comprises the steps of:
sequencing the exome of a tumour;
identifying tumor neoantigens comprising neoepitopes from said tumor;
selecting neoepitopes based on antigenicity,
   prior to the step of preparing the polynucleotide.

In a further aspect the present invention relates to a method of treating cancer in a patient, the method comprising administering to the patient in need thereof, the vaccine as defined herein.

Preferably, the vaccine is administered intradermally or intramuscular.

In an embodiment the nucleotide sequence is DNA. In another embodiment the nucleotide sequence is RNA.

In an preferred embodiment administration is carried out with a jet injector. Administration can for example be assisted by electroporation.

### DESCRIPTION OF DRAWINGS

FIG. 1 illustrates the total immune response in the B16 melanoma model mice injected with the DNA vaccine candidates NEO B16-X comprising 10 neoepitopes (VB4007, upper panel) or NEO B16-111 comprising 3 neoepitopes (VB4008, lower panel). The figure shows the total number of IFNγ-spots per 10⁶ splenocytes. Vaccines comprising 10 neoepitopes led to a stronger and broader total immune response than vaccines comprising 3 neoepitopes. As a negative control, mice were injected with empty vector not comprising the neoepitopes. As seen from the figure, injections with empty vector did not lead to any significant immune response.

NEO B16-X=VB4007=B16 pepM1-M10

NEO B16-III=VB4008=B16 pepM1-M3

FIG. 2 shows that injecting the B16 melanoma model mice with a mix of the corresponding 10 neoepitopes B16 pepM1-M10 plus adjuvant (either 20 or 200 μg peptide mix) only resulted in a vague immune response. As a negative control, mice were injected with empty vector not comprising the neoepitopes.

### DEFINITIONS

Tumor is used in the present context for both a solid tumor as well as for tumor cells found in a bodily fluid, such as blood.

Tumor neoantigen is used for any tumor specific antigen comprising one or more mutations as compared to the host's exome and is used synonymously with the term cancer neoantigen.

Tumor neoepitope is used for any immunogenic mutation in a tumor antigen and is used synonymously with the term cancer neoepitope.

Tumor neoepitope sequence is used to describe the sequence comprising the neoepitope in an antigenic subunit, and is used synonymously with the term cancer neoepitope sequence.

Therapeutic anticancer vaccine is used to describe that the vaccine is used for reducing or destroying tumor cells already present in the patient.

### DETAILED DESCRIPTION OF THE INVENTION

Cancers develop from the patient's normal tissue by one or a few cells starting an abnormal uncontrolled proliferation of the cells due to mutations. Although the cancer cells are mutated, most of the genome is intact and identical to the remaining cells in the patient. This is also the explanation of some of the failures in prior attempts to develop an anticancer vaccine, namely that the vaccine to some extent is also directed to the normal cells in the patient. As discussed above, the approach of attacking a tumor as defined by the present invention is to use the knowledge that any tumor, due to the mutations, expresses mutated proteins, so-called neoantigens that are not identical to any proteins in the normal cells of the patient, and therefore the neoantigens are efficient targets for a therapeutic anticancer vaccine. The mutations found in a tumor are normally highly individual, and accordingly, the vaccine according to the present invention is personalized for use only in the patient having the mutation in question.

The vaccines according to the present invention use the normal adaptive immune system to provide immunity against the tumor cells. The adaptive immune system is specific in that every foreign antigen evokes an immune response specifically towards said foreign antigen by the recognition of specific “non-self” antigens during a process called antigen presentation. The cells of the adaptive immune system are lymphocytes, in particularly B cells and T cells. B cells are involved in the humoral immune response, whereas T cells are involved in cell-mediated immune response.

In particularly, the vaccine according to the present invention is designed for evoking a cell-mediated immune response through activation of T cells against the neoantigens. T cells recognize neoepitopes when they have been processed and presented complexed to a MHC molecule as discussed below.

Major Histocompatibility Complex (MHC)

The neoepitopes according to the present invention are designed to be presented in MHC-neoepitope complexes. There are two primary classes of major histocompatibility complex (MHC) molecules, MHC I and MHC II.

MHC I is found on the cell surface of all nucleated cells in the body. One function of MHC I is to display peptides of non-self proteins from within the cell to cytotoxic T cells. The MHC I complex-peptide complex is inserted into the plasma membrane of the cell presenting the peptide to the cytotoxic T cells, whereby an activation of cytotoxic T cells against the particular MHC-peptide complex is triggered. The peptide is positioned in a groove in the MHC I molecule, allowing the peptide to be about 8-10 amino acids long.

MHC II molecules are a family of molecules normally found only on antigen-presenting cells such as dendritic cells, mononuclear phagocytes, some endothelial cells, thymic epithelial cells, and B cells.

As opposed to MHC I, the antigens presented by class II peptides are derived from extracellular proteins. Extracellular proteins are endocytosed, digested in lysosomes, and the resulting antigenic peptides are loaded onto MHC class II molecules and then presented at the cell surface. The antigen-binding groove of MHC class II molecules is open at both ends and is able to present longer peptides, generally between 15 and 24 amino acid residues long.

Class I MHC molecules are recognized by CD8 and co-receptors on the T cells, normally called CD8+ cells, whereas class II MHC molecules are recognized by CD4 and co-receptors on the T cells, normally called CD4+ cells.

Vaccines

The present invention relates to polynucleotide vaccines comprising cancer neoepitope sequences obtained by sequencing tumor DNA or RNA and identifying tumor specific mutations representing neoantigens. Thereby, a personalized neoantigen vaccine is obtained that specifically targets the identified tumor antigens.

Thus, in one aspect the present invention relates to a therapeutic anticancer vaccine comprising an immunologically effective amount of a polynucleotide comprising a nucleotide sequence encoding an antigenic unit comprising
from 2 to 50 antigenic subunits, each subunit comprising at least a part of a cancer neoepitope sequence and a linker and
a final cancer neoepitope sequence

The antigenic unit according to the invention comprises a plurality of tumor neoepitopes, wherein each neoepitope corresponds to a mutation identified in a tumor neoantigen. The mutation may be any mutation leading to a change in at least one amino acid. Accordingly, the mutation may be one of the following:
a non-synonymous mutation leading to a change in the amino acid
a mutation leading to a frame shift and thereby a completely different open reading frame in the direction after the mutation
a read-through mutation in which a stop codon is modified or deleted leading to a longer protein with a tumor-specific neoepitope
splice mutations that lead to a unique tumor-specific protein sequence
chromosomal rearrangements that give rise to a chimeric protein with a tumor-specific neoepitope at the junction of the two proteins

In the antigenic unit, all but the last of the tumor neoepitopes are arranged in antigenic subunits, wherein each subunit consists of a tumor neoepitope sequence and a linker, whereas the last subunit comprises a neoepitope only, i.e. no such linker. Due to the separation of the tumor neoepitope sequences by said linker, each neoepitope is presented in an optimal way to the immune system, whereby the efficiency of the vaccine is ensured as discussed below.

In one embodiment, depending on the selected neoepitopes, the antigenic subunits are arranged in the order of more antigenic to less antigenic.

The cancer neoepitope sequence preferably has a length suitable for presentation by the MHC molecules discussed above. Thus, in a preferred embodiment the cancer neoepitope is from 7 to 30 amino acids long. More preferred are cancer neoepitope sequences having a length of from 7 to 10 amino acids or cancer neoepitope sequences having a length of from 13 to 30 amino acids.

In order to avoid that tumors escape the immune system by shutting down expression of a mutated gene if the vaccine is directed towards the expression product of said gene, it is preferred to include a plurality of different neoepitopes into the antigenic unit. In general the more genes the tumor has to shut down the less likely is it that the tumor is capable of shutting down all of them and still be able to proliferate or even survive. Furthermore, the tumor may be heterogeneous in that not each and every neoantigen is expressed by all the tumor cells. Accordingly, in accordance with the present invention, the approach is to include as many neoepitopes as possible into the vaccine in order to attack the tumor efficiently. Also, in order to secure that all neoepitopes are loaded efficiently to the same antigen presenting cell they are arranged as one amino acid chain instead of as discrete peptides. However, as described above, the object of the vaccine is to activate the T cells against the neoepitopes, and the T cells may be diluted in case too many neoepitopes are included into the vaccine, and therefore it is a balance to provide the vaccine with an optimal number of neoepitopes in the antigenic unit.

As discussed below in more details, the tumor exome is analysed to identify neoantigens and subsequently the most antigenic neoepitopes are selected. The present inventor has found that at least 3 neoepitopes should be selected to be incorporated into the vaccine, such as at least 5 neoepitopes, such as at least 7 neoepitopes, such as at least 10 neoepitopes, in order to efficiently be able to “hit” substantially all tumor cells.

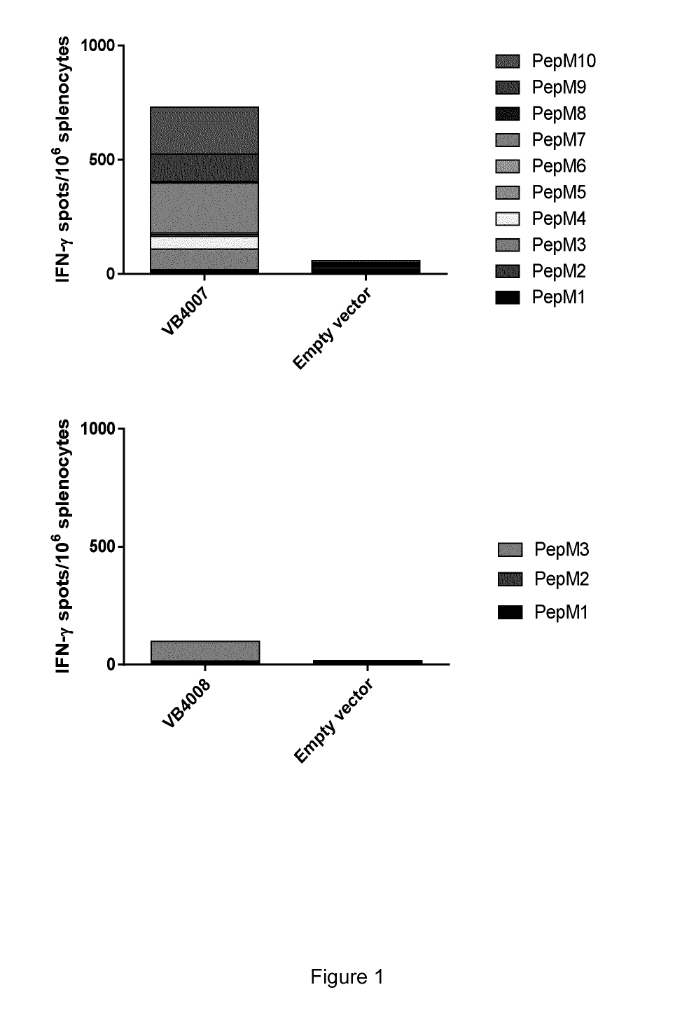

In addition, the inventors of the present invention have found that the increasing the numbers of neoepitopes in the vaccine constructs from 3 neoepitopes to 10 neoepitopes leads to a surprising increase in the immune response (see FIG. 1). Preferably, the vaccine according to the present invention comprises at least 10 neoepitopes.

In one embodiment from 3 to 50 neoepitopes are included in the vaccine in order to obtain the most efficient immune response without diluting the T cells, such as from 3 to 30 neoepitopes, such as from 3 to 20 neoepitopes, such as from 3 to 15 neoepitopes, such as from 3 to 10 neoepitopes, and consequently n is preferably an integer of from 3 to 50, such as from 3 to 30, such as from 5 to 25, such as from 3 to 20, such as from 3 to 15, such as from 3 to 10.

In another embodiment 5 to 50 neoepitopes may be included in the vaccine in order to obtain the most efficient immune response without diluting the T cells, such as from 5 to 30 neoepitopes, such as for example from 5 to 25 neoepitopes, such as from 5 to 20 neoepitopes, such as from 5 to 15 neoepitopes, such as from 5 to 10 neoepitopes, and consequently n is preferably an integer of from 5 to 50, such as from 5 to 30, such as from 5 to 20, such as from 5 to 15, such as from 5 to 10.

In a further embodiment 10 to 50 neoepitopes may be included in the vaccine in order to obtain the most efficient immune response without diluting the T cells, such as from 10 to 40 neoepitopes, such as from 10 to 30 neoepitopes, such as from 10 to 25 neoepitopes, such as from 10 to 20 neoepitopes, such as from 10 to 15 neoepitopes, and consequently n is preferably an integer of from 10 to 50, such as from 10 to 30, such as for example from 10 to 25, such as from 10 to 20 or such as from 10 to 15 neoepitopes.

The inventors of the present invention have shown that vaccibody DNA vaccines comprising 10 neoepitopes induces a stronger and broader total immune response than vaccibody DNA vaccines comprising only 3 neoepitopes (see FIG. 4 and Example 2). Further, increasing the number of neoepitopes to more than 20 may result in a less efficient vaccine due to a dilution of the T cells. Further, it can be associated with technical difficulties to include more than 20 neoepitopes.

Accordingly, in a preferred embodiment of the present invention the vaccine comprises from 10 to 20 neoepitopes.

In yet another embodiment 15 to 50 neoepitopes are included in the vaccine in order to obtain the most efficient immune response without diluting the T cells, such as from 15 to 30 neoepitopes or such as from 15 to 20 neoepitopes and consequently n is preferably an integer of from 15 to 50, such as from 15 to 30 or such as from 15 to 20 neoepitopes.

In one embodiment, the antigenic unit comprises one copy of each cancer neoepitope, so that when 10 neoepitopes are included in the vaccine a cell-mediated immune response against 10 different neoepitopes can be evoked.

If however only a few relevant antigenic mutations are identified, then the antigenic unit may comprise at least two copies of at least one neoepitope in order to strengthen the immune response to these neoepitopes. Also for manufacturing and regulatory reasons it may be an advantage to keep the length of plasmid and i.e. the antigenic unit constant, and therefore it may be advantageously to include more than one copy of the same neoepitope in the antigenic unit.

As discussed above, it may be an advantage to keep the length of the antigenic unit constant, and therefore it is preferred in one embodiment that all the cancer neoepitope sequences have identical length. However, if one or more of the neoepitopes result from a mutation leading to a frame shift or stop codon mutation, the neoepitope may have a substantial length, such as consisting of at least the mutated part of the protein, the most antigenic portion of the mutated protein or maybe of the whole mutated protein, whereby the length of at least one of the neoepitopes is substantially longer than the neoepitopes arising from a non-synonymous point mutation.

The length of the antigenic unit is primarily determined by the length of the neoepitopes and the number of neoepitopes arranged in the antigenic unit and is from about 21 to 1500, preferably from about 30 amino acids to about a 1000 amino acids, more preferably from about 50 to about 500 amino acids, such as from about 100 to about 400 amino acids, from about 100 to about 300 amino acids.

In particularly when the neoepitope is short, such as a few amino acids long, the cancer neoepitope sequence comprises the neoepitope flanked at both sides by an amino acid sequence. Preferably, the neoepitope is positioned essentially in the middle of a cancer neoepitope sequence, in order to ensure that the neoepitope is presented by the antigen presenting cells after processing. The amino acid sequences flanking the neoepitope are preferably the amino acid sequences flanking the neoepitope in the neoantigen, whereby the cancer neoepitope sequence is a true subsequence of the cancer neoantigen amino acid sequence.

Although it is possible to obtain a relevant immune response towards the tumor if the neoepitopes are randomly arranged in the antigenic subunit, it is preferred to follow at least one of the following methods for ordering the neoepitopes in the antigenic unit in order to enhance the immune response.

In another embodiment, in particularly if the hydrophilicity/hydrophobicity varies greatly among the neoepitopes, it is preferred that the most hydrophobic antigenic subunit(s) is(are) substantially positioned in the middle of the antigenic unit and the most hydrophilic antigenic subunit(s) is/are positioned at the beginning and/or end of the antigenic unit. Alternatively, the neoepitopes may be arranged alternating between a hydrophilic and a hydrophobic neoepitope.

Furthermore, GC rich neoepitopes should be spaced so that GC clusters are avoided; preferably GC rich neoepitopes are spaced by at least one subunit.

The linker is designed to be non-immunogenic and is preferably also a flexible linker, whereby the tumor neoepitopes, in spite of the high numbers of antigenic subunits present in the antigenic unit, are presented in an optimal manner to the T cells. Preferably, the length of the linker is from 4 to 20 amino acids to secure the flexibility. In another preferred embodiment, the length of the linker is from 8 to 20 amino acids, such as from 8 to 15 amino acids, for example 8 to 12 amino acids or such as for example from 10 to 15 amino acids. In a particular embodiment, the length of the linker is 10 amino acids.

In a specific embodiment, the vaccine of the present invention comprises 10 neoepitopes wherein the linkers have a length of from 8 to 20 amino acids, such as from 8 to 15 amino acids, for example 8 to 12 amino acids or such as for example from 10 to 15 amino acids. In a particular embodiment, the vaccine of the present invention comprises 10 neoepitopes wherein the linkers, have a length of 10 amino acids.

The linker is preferably identical in all antigenic subunits. If, however, one or more of the neoepitopes comprise an amino acid motif similar to the linker, it may be an advantage to substitute the neighbouring linkers with a linker of a different sequence. In addition, if a neoepitope-second linker junction is predicted to constitute an epitope in itself, then a linker of a different sequence might be used.

The linker is preferably a serine-glycine linker, such as a flexible GGGGS linker, such as GGGSS, GGGSG, GGGGS or multiple variants thereof such as GGGGSGGGGS or (GGGGS)ₘ, (GGGSS)ₘ, (GGGSG)ₘ, where m is an integer from 1 to 5, from 1 to 4 or from 1 to 3. In a preferred embodiment m is 2.

In a preferred embodiment the serine-glycine linker further comprises at least one leucine (L), such as at least 2 or at least 3 leucines. The serine-glycine linker may for example comprise 1, 2, 3 or 4 leucines. Preferably, the serine-glycine linker comprises 1 leucine or 2 leucines.

In one embodiment the second linker comprises or consists of the sequence LGGGS, GLGGS, GGLGS, GGGLS or GGGGL. In another embodiment the second linker comprises or consists of the sequence LGGSG, GLGSG, GGLSG, GGGLG or GGGSL. In yet another embodiment the second linker comprises or consists of the sequence LGGSS, GLGSS, GGLSS, GGGLS or GGGSL.

In yet another embodiment the second linker comprises or consists of the sequence LGLGS, GLGLS, GLLGS, LGGLS or GLGGL. In another embodiment the second linker comprises or consists of the sequence LGLSG, GLLSG, GGLSL, GGLLG or GLGSL. In yet another embodiment the second linker comprises or consists of the sequence LGLSS, GLGLS, GGLLS, GLGSL or GLGSL.

In another embodiment of the present invention the second serine-glycine linker has a length of 10 amino acids and comprises 1 leucine or 2 leucines.

In one embodiment the second linker comprises or consists of the sequence LGGGSGGGGS, GLGGSGGGGS, GGLGSGGGGS, GGGLSGGGGS or GGGGLGGGGS. In another embodiment the second linker comprises or consists of the sequence LGGSG GGGSG, GLGSGGGGSG, GGLSGGGGSG, GGGLGGGGSG or GGGSLGGGSG. In yet another embodiment the second linker comprises or consists of the sequence LGGSSGGGSS, GLGSSGGGSS, GGLSSGGGSS, GGGLSGGGSS or GGGSLGGGSS.

In a further embodiment the second linker comprises or consists of the sequence LGGGSLGGGS, GLGGSGLGGS, GGLGSGGLGS, GGGLSGGGLS or GGGGLGGGGL. In another embodiment the second linker comprises or consists of the sequence LGGSGLGGSG, GLGSGGLGSG, GGLSGGGLSG, GGGLGGGGLG or GGGSLGGGSL. In yet another embodiment the second linker comprises or consists of the sequence LGGSSLGGSS, GLGSSGLGSS, GGLSSGGLSS, GGGLSGGGLS or GGGSLGGGSL.

In a preferred embodiment the vaccine according to the present invention comprises at least 10 neoepitopes that are separated by 10 amino acid linkers. In another preferred embodiment the vaccine according to the present invention comprises at least 15 neoepitopes that are separated by 10 amino acid linkers, such as at least 20 neoepitopes that are separated by 10 amino acid linkers.

In another preferred embodiment the vaccine comprises from 10 to 20 or from 10 to 25 neoepitopes that are separated by second linkers. Preferably, said second linkers are 10 amino acids. The second linker may also have any length as defined herein above, such as for example from 8 to 12 amino acids.

Alternative linkers may be selected from the group consisting of GSAT linkers and SEG linkers, or multiple variants thereof.

Signal Peptide

In a preferred embodiment, the polynucleotide further comprises a nucleotide sequence encoding a signal peptide. The signal peptide is constructed to allow secretion of the polypeptide encoded by the polynucleotide of the invention in the cells transfected with said polynucleotide.

Any suitable signal peptide may be used. Examples of suitable peptides are an Ig VH signal peptide, such as SEQ ID NO: 31, a human TPA signal peptide, such as SEQ ID NO: 32, and a signal peptide comprising an amino acid sequence having at least 80% sequence identity to the amino acid sequence 1-23 of SEQ ID NO:1.

In a preferred embodiment the signal peptide comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to the amino acid sequence 1-23 of SEQ ID NO:1.

In a more preferred embodiment, the signal peptide consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to the amino acid sequence 1-23 of SEQ ID NO:1.

Sequence Identity

Sequence identity may be determined as follows: A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalise to gaps in the end of the sequences. When utilizing the ALIGN og Align0 program for comparing amino acid sequences, a BLOSUM50 substitution matrix with gap opening/extension penalties of −12/−2 is preferably used.

Polynucleotides

The invention also relates to a polynucleotide as described above. The polynucleotide may comprise a DNA nucleotide sequence or a RNA nucleotide sequence, such as genomic DNA, cDNA, and RNA sequences, either double stranded or single stranded.

It is preferred that the polynucleotide is optimized to the species to express the polypeptide according to the invention, i.e. it is preferred that the polynucleotide sequence is human codon optimized.

Polypeptides

The invention further relates to a polypeptide encoded by the polynucleotide sequence as defined above. The polypeptide may be expressed in vitro for production of the vaccine according to the invention, or the polypeptide may be expressed in vivo as a result of administration of the polynucleotide as defined above.

Vector

Furthermore, the invention relates to a vector comprising a nucleotide sequence as defined above. It is preferred that the vector allows for easy exchange of the various units described above, in particularly the antigenic unit. In particularly, the expression vector may be pUMVC4a vector or NTC9385R vector backbones. The antigenic unit may be exchanged with an antigenic unit cassette restricted by the Sfil restriction enzyme cassette where the 5′ site is incorporated in the GLGGL/GLSGL linker and the 3′ site is included after the stop codon in the vector.

Host Cell

The invention also relates to a host cell comprising a nucleotide sequence as defined above or comprising a vector as defined above for expression of the polypeptide according to the invention.

Suitable host cells include prokaryotes, yeast, insect or higher eukaryotic cells.

Methods for Preparing the Vaccine

The vaccine according to the invention is preferably a personalized vaccine in the sense that the neoantigens are identified in the patient's tumor and accordingly, the vaccine is directed exactly against the specific mutated proteins in the patient's tumor.

Accordingly, in one aspect the invention relates to a method for preparing a vaccine comprising an immunologically effective amount of the polypeptide as defined above by producing the polypeptides in vitro. The in vitro synthesis of the polypeptides and proteins may be carried out by any suitable method known to the person skilled in the art, such as through peptide synthesis or expression of the polypeptide in any of a variety of expressions systems followed by purification. Accordingly, in one embodiment the method comprises
a) transfecting the polynucleotide as defined above into a cell population;
b) culturing the cell population;
c) collecting and purifying the polypeptide expressed from the cell population, and
d) mixing the polypeptide obtained under step c) with a pharmaceutically

In a preferred embodiment, the dimeric protein or polypeptide obtained under step c) is dissolved in said pharmaceutically acceptable carrier.

Furthermore, an adjuvant or buffer may be added to the vaccine.

Purification may be carried out according to any suitable method, such as chromatography, centrifugation, or differential solubility.

In another aspect, the invention relates to a method for preparing a vaccine comprising an immunologically effective amount of the polynucleotide as defined above. In one embodiment the method comprises
a. preparing the polynucleotide as defined above;
b. mixing the polynucleotide obtained under step a) with a pharmaceutically acceptable carrier thereby obtaining the vaccine.

The polynucleotide may be prepared by any suitable method known to the skilled person. For example, the polynucleotide may be prepared by chemical synthesis using an oligonucleotide synthesizer.

In particularly, smaller nucleotide sequences, such as for example subunits of the antigenic unit may be synthesized individually and then ligated to produce the final polynucleotide into the vector backbone.

For the design of a personalized vaccine, the methods above are preceded by a method of identifying the neoepitopes to be included into the polynucleotide.

This method preferably includes the steps of
sequencing the genome, or exome of a tumor
identifying tumor neoantigens comprising neoepitopes from said tumor,
selecting neoepitopes based on predicted antigenicity.

The tumor or tumor part may be by through any suitable method, such as by obtaining a biopsy of the tumor or by excision of the tumor, or from any suitable body fluid, such as a blood sample or a urine sample.

Sequencing of Tumor Genome or Exome

The genome or the exome, i.e. the coding part of the genome, may be sequenced using any suitable method, such as whole exome sequencing. In particularly the sequencer may be an Illumina HiSeq2500), using Paired-end 2×100-125 or PE100-125 (read length), multiplex.

Identifying Tumor Antigens

Once the tumor specific mutations are identified, the next step is to identify predicted antigenic peptides comprising the neoepitopes.

Tumor mutations are discovered by sequencing of tumor and normal tissue and make a comparison of the obtained sequences. A variety of methods is available for detecting the presence of a particular mutation or allele in an individual's DNA or RNA. For example techniques including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips may be applied.

Alternatively, a method for identifying mutations by direct protein sequencing may be carried out.

Out of the maybe hundreds or thousands of mutations in the tumor exome, the neoepitopes are selected in silico based on predictive HLA-binding algorithms. The intention is to identify all relevant neoepitopes and after a ranking or scoring determine the neoepitopes to be included in the vaccine for the specific patient in question.

Any suitable algorithms may be used, such as one of the following:

Available free software analysis of peptide-MHC binding (IEDB and NetMHC) may be downloaded from the following websites:

http://www.iedb.org/

http://www.cbs.dtu.dk/services/NetMHC/

Commercially available advanced software to predict optimal peptides for vaccine design are e.g. found here:

http://www.oncoimmunity.com/

https://omictools.com/t-cell-epitopes-category

https://github.com/griffithlab/pVAC-Seq

http://crdd.osdd.net/raghava/cancertope/help.php

http://www.epivax.com/tag/neoantigen

Each mutation is scored with respect to its antigenicity, and the most antigenic neoepitopes are selected and optimally designed in the polynucleotide. As discussed above from 3 to 50 neoepitopes are preferred according to the present invention.

Vaccine

The final vaccine is then produced to comprise one of the following:
the polynucleotide as defined above
the polypeptide encoded by the polynucleotide as defined above

The vaccine may further comprise a pharmaceutically acceptable carrier, diluent, adjuvant or buffer.

Pharmaceutically acceptable carriers, diluents, and buffers include, but are not limited to, saline, buffered saline, dextrose, water, glycerol, ethanol, sterile isotonic aqueous buffer, and combinations thereof.

In particularly for vaccines comprising polypeptides/proteins pharmaceutically acceptable adjuvants include, but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1P321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, resiquimod, SRL172, Virosomes and other Virus-like particles, YF-17D, VEGF trap, R848, beta-glucan, Pam3Cys, Aquila's QS21 stimulon, vadimezan, and/or AsA404 (DMXAA).

In particularly for vaccines comprising polynucleotides the carriers may include molecules that ease transfection of cells and adjuvants may include plasmids comprising nucleotide sequences encoding chemokines or cytokines in order to enhance the immune response.

The vaccine is formulated into any suitable formulation, such as a liquid formulation for intradermal or intramuscular injection.

Administration

The vaccine may be administered in any suitable way for either a polypeptide/protein vaccine or a polynucleotide vaccine, such as administered by injection intradermally, intramuscular, subcutaneously, or by mucosal or epithelial application, such as intranasally, orally, enteral or to the bladder. In one preferred embodiment, the vaccine is administered by jet-injection.

In particularly the vaccine is preferably administered intramuscular or intradermally when the vaccine is a polynucleotide vaccine.

In a specific embodiment the vaccine is administered by intranodal injection. As used herein, the term “intranodal injection” means that the vaccine is injected into the lymph nodes.

Treatment

The polynucleotides and polypeptides are preferably for use in the treatment of cancer, and formulated in a vaccine as discussed above. By the methods described herein it is possible to treat a patient suffering from cancer by examining any mutations present in the tumor in the patient, producing the vaccine and then immunizing the patient with the vaccine directed exactly to neoantigens present in his or her tumor. Due to the fast and reliable methods for sequencing, epitope-determining and producing nucleotide sequences today, it has become likely that a patient may receive the vaccine within 12 weeks from having the tumor resected

The cancer may be any cancer wherein the cancer cells comprise mutations. The cancer may be a primary tumor, metastasis or both. The tumor examined for mutations may be a primary tumor or a metastasis. The cancers to be treated are in particularly the cancers known to have a high mutational load, such as melanomas, lung cancer, breast cancer, prostate cancer and colon cancer.

In a preferred embodiment the treatment is performed with a vaccine comprising a polynucleotide as described above, for example wherein the polynucleotide is DNA or RNA.

It is preferred to inject a polynucleotide vaccine intramuscular, such as in the big muscles, for example in the shoulder, buttock or thigh. It has been found that the polypeptides are produced locally and relevant immune cells internalize the polypeptides/proteins essentially at the site of production, and substantially no polypeptides or proteins reach the blood stream.

Any suitable method for injecting the polynucleotide may be used, such as by the use of a jet injector or assisted by electroporation.

Dosage Regimen

The vaccine may be administered as a single dosage, or may be repeated. When the vaccine administration is repeated it is preferred that it is administered with at least 3-week intervals, to avoid exhaustion of the T cells.

Accordingly, in one embodiment the dosage regimen would be vaccination week 0, 3, 6 and then every 4 weeks as long as the patient has clinical benefit. The vaccine may be administered for at least a year.

The vaccine is administered in an immunologically effective amount. By “immunologically effective amount” is meant the amount of the vaccine required to establish a tumor reducing effect. Ultimately, the physician determines the dosage that typically is in the range of 0.3-6 mg for DNA vaccines, and in the range of 5 μg-5 mg for polypeptide/protein vaccines.

Combination Treatments

The vaccine treatment according to the present invention may be combined with any other anticancer treatment, such as radiation therapy, chemotherapy, and surgical treatment.

The vaccine treatment according to the invention may also be combined with checkpoint-blockade inhibitor treatment.

### EXAMPLES

### Example 1

### Construction and Expression of the Vaccines

Previously described exome sequencing and RNA sequencing of the mouse melanoma cancer cell line B16-F10 revealed hundreds to thousands of tumor-specific non-synonymous mutations (Castle et al 2012, Castle et al 2014 and Kreiter et al 2015). In silico-based methods were used to identify potential immunogenic neo-epitopes. Mice were immunized with peptides encoding the mutated epitopes, and their immunogenicity was observed as specific T cell immune responses (Elispot assay). Furthermore, vaccination of mice with the most immunogenic epitopes selected from the Elispot conferred strong anti-tumor activity (Castle et al 2012 and Kreiter et al 2015).

Each of the neo-epitopes are peptides of 27 amino acids separated by a flexible GGGGS linker. Short peptides (<20 amino acids) are processed and novel epitopes may be presented on MHC class I molecules and activate CD8+ T cells. However, it is preferred that the vaccine activates CD8+ and CD4+ T cells and therefore neo-epitopes encoding for long peptides (>20 amino acids) are chosen. That may allow for efficient peptide processing and presentation on both MHC class I and II (Kreiter et al 2015). In the NEO B16-X construct, the selected hydrophobic and hydrophilic neo-epitopes are evenly distributed. A neutral, flexible GGGGS linker between the 27mer neo-epitopes is important to avoid generation of new immunogenic epitopes in the junctions of the combined neo-epitopes.

The sequences of the neoepitopes found in the B16-F10 cell line is shown in Table 1.

TABLE 1
B16-F10 cell line

| Muta- | | | | | |
|---|---|---|---|---|---|
| tion | | | | | |
| number | | | | Reac- | MHC I |
| poly- | | | | tive | score |
| pep- | | Mutated | | T | (best |
| tide | | sequence | Substi. | cell | pre- |
| (Vacci- | | used for | WT, AA | sub- | dic- |
| body) | Gene | vaccination | #, Mut) | type | tion) |
| B16- | Kif18b | PSKPSFQEFVDWENV | K739N | CD4+ | 1, 2 |
| PepM1 | | SPELNSTDQPFL | | | |
| | | (SEQ ID NO: 1) | | | |
| B16- | Obsl1 | REGVELCPGNKYEMR | T176M | CD8+ | 2, 3 |
| PepM2 | | RHGTTHSLVIHD | | | |
| | | (SEQ ID NO: 2) | | | |
| B16- | Def8 | SHCHWNDLAVIPAGV | R255G | CD4+ | 3, 8 |
| PepM3 | | VHNWDFEPRKVS | | | |
| | | (SEQ ID NO: 3) | | | |
| B16- | Rpl13a | GRGHLLGRLAAIVGK | A24G | CD4+ | 0, 5 |
| PepM4 | | QVLLGRKVVVVR | | | |
| | | (SEQ ID NO: 4) | | | |
| B16- | Tubb3 | FRRKAFLHWYTGEAM | G402A | CD4+ | 1, 9 |
| PepM5 | | DEMEFTEAESNM | | | |
| | | (SEQ ID NO: 5) | | | |
| B16- | Tnpo3 | VVDRNPQFLDPVLAY | G504A | CD4+ | 1 |
| PepM6 | | LMKGLCEKPLAS | | | |
| | | (SEQ ID NO: 6) | | | |
| B16- | Atp11a | SSPDEVALVEGVQSL | R552S | CD4+ | 0, 1 |
| PepM7 | | GFTYLRLKDNYM | | | |
| | | (SEQ ID NO: 7) | | | |
| B16- | Cpsf3I | EFKHIKAFDRTFANN | D314N | CD4+ | 0, 5 |
| PepM8 | | PGPMVVFATPGM | | | |
| | | (SEQ ID NO: 8) | | | |
| B16- | Plod1 | STANYNTSHLNNDVW | F530V | CD4+ | 0, 1 |
| PepM9 | | QIFENPVDWKEK | | | |
| | | (SEQ ID NO: 9) | | | |
| B16- | Pbk | DSGSPFPAAVILRDA | V145D | CD8+ | 0, 1 |
| PepM10 | | LHMARGLKYLHQ | | | |
| | | (SEQ ID NO: 10) | | | |

All neoepitope gene sequences were ordered from Genscript (New Jersey, US) and cloned into the expression vector pUMVC4a.

### Example 2

### Immune Response Studies

NEO B16-X comprising 10 neoepitopes (VB4007) and NEO B16-III comprising 3 neoepitopes (VB4008) were selected as vaccine candidates. As a negative control, empty pUMVC4a vector was utilized.

NEO B16-X=VB4007=B16 pepM1-M10

NEO B16-III=VB4008=B16 pepM1-M3

The neoepitope sequences used for the DNA vaccines are shown in Table 1.

20 μg plasmid DNA of each candidate were injected intramuscularly in the tibialis anterior muscle of C57B1/6 mice followed by electroporation using TriGrid, Ichor, (US). At day 13, the mice were euthanized and spleens were harvested.

The T cell responses were evaluated by IFN-gamma ELISpot. The results are shown in FIG. 1 where the total neoantigen-specific T-cell response is indicated as the number of IFN-γ spots/10⁶ splenocytes. It was observed that the DNA vaccine comprising the neoepitopes B16-pepM1-pepM10 (VB4007) led to a strong immune response. Injection with empty vector did not result in a significant immune response (Upper panel, FIG. 1). NEO B16-III comprising 3 neoepitopes (VB4008) also induces a neoepitope specific immune response, although a weaker total immune response is observed compared to VB4007 (Lower panel, FIG. 1).

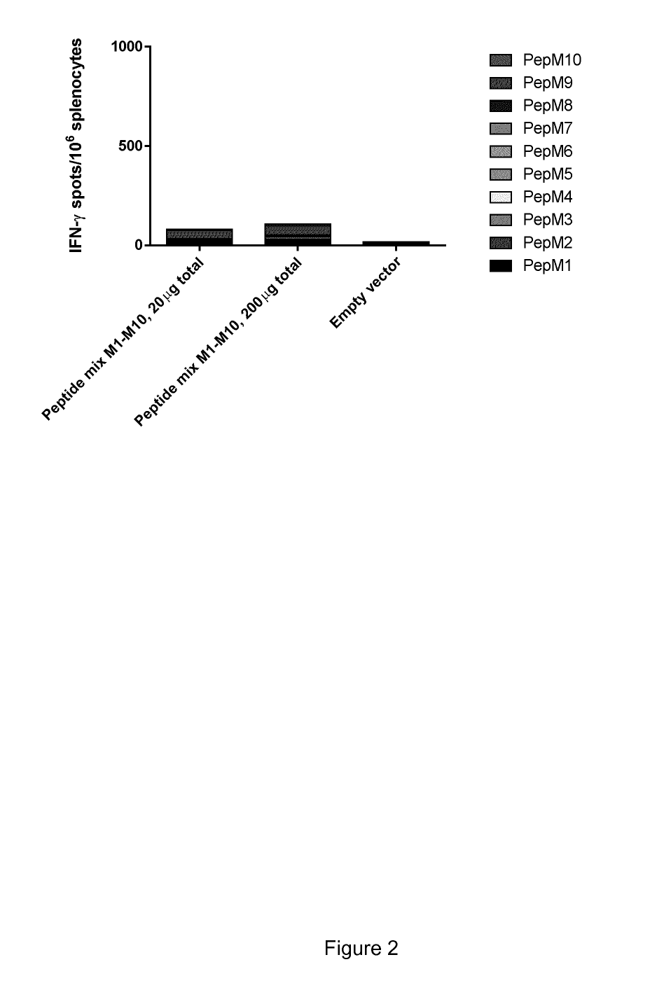

As a further control, mice were injected subcutaneoulsy with a mix of the 10 corresponding peptides B16-pepM1-pepM10 plus 50 μg poly (I:C) adjuvant. FIG. 2 shows the results of experiments where mice were injected with either 20 or 200 μg peptides mix plus adjuvant (i.e. 2 μg or 20 μg of each individual neoepitope). Injecting mice with a peptide mix plus adjuvant comprising the same 10 neoepitope sequences as present in the NEO B16-X construct (VB4007) did not result in any significant immune response compared to the immune response induced by VB4007 (as shown in FIG. 1 upper panel).

### Example 4

A therapeutic DNA vaccine to be used may be prepared by GMP manufacturing of the plasmid vaccine according to regulatory authorities' guidelines, and Fill & Finish of the DNA vaccine. The DNA vaccine may be formulated by dissolving in a saline solution, such as PBS at a concentration of 2-6 mg/ml. The vaccine may be administered either intradermal or intramuscular with or without following electroporation or alternatively with a jet injector.

SEQUENCES
B16-F10 mutated epitope, B16-PepM1, amino acid
sequence
SEQ ID NO: 1
PSKPSFQEFVDWENVSPELNSTDQPFL
B16-F10 mutated epitope, B16-PepM2, amino acid
sequence
SEQ ID NO: 2
REGVELCPGNKYEMRRHGTTHSLVIHD
B16-F10 mutated epitope, B16-PepM3, amino acid
sequence
SEQ ID NO: 3
SHCHWNDLAVIPAGVVHNWDFEPRKVS
B16-F10 mutated epitope, B16-PepM4, amino acid
sequence
SEQ ID NO: 4
GRGHLLGRLAAIVGKQVLLGRKVVVVR
B16-F10 mutated epitope, B16-PepM5, amino acid
sequence
SEQ ID NO: 5
FRRKAFLHWYTGEAMDEMEFTEAESNM
B16-F10 mutated epitope, B16-PepM6, amino acid
sequence
SEQ ID NO: 6
VVDRNPQFLDPVLAYLMKGLCEKPLAS
B16-F10 mutated epitope, B16-PepM7, amino acid
sequence
SEQ ID NO: 7
SSPDEVALVEGVQSLGFTYLRLKDNYM
B16-F10 mutated epitope, B16-PepM8, amino acid
sequence
SEQ ID NO: 8
EFKHIKAFDRTFANNPGPMVVFATPGM
B16-F10 mutated epitope, B16-PepM9, amino acid
sequence
SEQ ID NO: 9
STANYNTSHLNNDVWQIFENPVDWKEK
B16-F10 mutated epitope, B16-PepM10, amino acid
sequence
SEQ ID NO: 10
DSGSPFPAAVILRDALHMARGLKYLHQ
SEQ ID NO: 11
Gly-Ser Linker: GGGGS
Amino acid sequence of VB4007 = NEO B16-X c B16
pepM1-M10, 5 aa linker
The neoepitope sequences start at amino acid 24.
The neoepitope sequences are separated by the
linkers GGGGS. Amino acids 1-24 represent the
signal peptide (SEQ ID NO: 14).
SEQ ID NO: 12
MQVSTAALAVLLCTMALCNQVLSPSKPSFQEFVDWENVSPELNSTDQPFL
GGGGSREGVELCPGNKYEMRRHGTTHSLVIHDGGGGSSHCHWNDLAVIPA
GVVHNWDFEPRKVSGGGGSGRGHLLGRLAAIVGKQVLLGRKVVVVRGGGG
SFRRKAFLHWYTGEAMDEMEFTEAESNMGGGGSVVDRNPQFLDPVLAYLM
KGLCEKPLASGGGGSSSPDEVALVEGVQSLGFTYLRLKDNYMGGGGSEFK
HIKAFDRTFANNPGPMVVFATPGMGGGGSSTANYNTSHLNNDVWQIFENP
VDWKEKGGGGSDSGSPFPAAVILRDALHMARGLKYLHQ
Amino acid sequence of VB4008 = NEO B16-III = B16
pepM1-M3, 5 aa linker
The neoepitope sequences start at amino acid 24.
The neoepitope sequences are separated by the
linkers GGGGS. Amino acids 1-24 represent the
signal peptide (SEQ ID NO: 14).
SEQ ID NO: 13
MQVSTAALAVLLCTMALCNQVLSPSKPSFQEFVDWENVSPELNSTDQPFL
GGGGSREGVELCPGNKYEMRRHGTTHSLVIHDGGGGSSHCHWNDLAVIPA
GVVHNWDFEPRKVS
Signal peptide
SEQ ID NO: 14
MQVSTAALAVLLCTMALCNQVLS
SEQ ID NO: 15
Linker: GGGSS.
SEQ ID NO: 16
Linker: GGGSG.
SEQ ID NO: 17
Linker: GGGGS.
SEQ ID NO: 18
Linker: LGGGS.
SEQ ID NO: 19
Linker: GLGGS.
SEQ ID NO: 20
Linker: GGLGS.
SEQ ID NO: 21
Linker: GGGLS.
SEQ ID NO: 22
Linker: GGGGL.
SEQ ID NO: 23
Linker: LGGSG.
SEQ ID NO: 24
Linker: GLGSG.
SEQ ID NO: 25
Linker: GGLSG.
SEQ ID NO: 26
Linker: GGGLG.
SEQ ID NO: 27
Linker: GGGSL.
SEQ ID NO: 28
Linker: LGGSS.
SEQ ID NO: 29
Linker: GLGSS.
SEQ ID NO: 30
Linker: GGLSS.
SEQ ID NO: 31
Linker: GGGLS.
SEQ ID NO: 32
Linker: GGGSL.
SEQ ID NO: 33
Linker: LGLGS.
SEQ ID NO: 34
Linker: GLGLS.
SEQ ID NO: 35
Linker: GLLGS.
SEQ ID NO: 36
Linker: LGGLS.
SEQ ID NO: 37
Linker: GLGGL.
SEQ ID NO: 38
Linker: LGLSG.
SEQ ID NO: 39
Linker: GLLSG.
SEQ ID NO: 40
Linker: GGLSL.
SEQ ID NO: 41
Linker: GGLLG.
SEQ ID NO: 42
Linker: GLGSL.
SEQ ID NO: 43
Linker: LGLSS.
SEQ ID NO: 44
Linker: GLGLS.
SEQ ID NO: 45
Linker: GGLLS.
SEQ ID NO: 46
Linker: GLGSL.
SEQ ID NO: 47
Linker: GLGSL.
SEQ ID NO: 48
Linker: LGGGSGGGGS.
SEQ ID NO: 49
Linker: GLGGSGGGGS.
SEQ ID NO: 50
Linker: GGLGSGGGGS.
SEQ ID NO: 51
Linker: GGGLSGGGGS.
SEQ ID NO: 52
Linker: GGGGLGGGGS.
SEQ ID NO: 53
Linker: LGGSGGGGSG.
SEQ ID NO: 54
Linker: GLGSGGGGSG.
SEQ ID NO: 55
Linker: GGLSGGGGSG.
SEQ ID NO: 56
Linker: GGGLGGGGSG.
SEQ ID NO: 57
Linker: GGGSLGGGSG.
SEQ ID NO: 58
Linker: GGGSLGGGSG.
SEQ ID NO: 59
Linker: GLGSSGGGSS.
SEQ ID NO: 60
Linker: GGLSSGGGSS.
SEQ ID NO: 61
Linker: GGGLSGGGSS.
SEQ ID NO: 62
Linker: GGGSLGGGSS.
SEQ ID NO: 63
Linker: LGGGSLGGGS.
SEQ ID NO: 64
Linker: GLGGSGLGGS.
SEQ ID NO: 65
Linker: GGLGSGGLGS.
SEQ ID NO: 66
Linker: GGGLSGGGLS.
SEQ ID NO: 67
Linker: GGGGLGGGGL.
SEQ ID NO: 68
Linker: LGGSGLGGSG.
SEQ ID NO: 69
Linker: GLGSGGLGSG.
SEQ ID NO: 70
Linker: GGLSGGGLSG.
SEQ ID NO: 71
Linker: GGGLGGGGLG.
SEQ ID NO: 72
Linker: GGGSLGGGSL.
SEQ ID NO: 73
Linker: LGGSSLGGSS.
SEQ ID NO: 74
Linker: GLGSSGLGSS.
SEQ ID NO: 75
Linker: GGLSSGGLSS.
SEQ ID NO: 76
Linker: GGGLSGGGLS.
SEQ ID NO: 77
Linker: GGGSLGGGSL.

1. A therapeutic anticancer vaccine comprising an immunologically effective amount of a polynucleotide comprising a nucleotide sequence encoding an antigenic unit comprising
from 2 to 50 antigenic subunits, each subunit comprising at least a part of a cancer neoepitope sequence and a linker and
a final cancer neoepitope sequence.

2. The vaccine according to claim 1, wherein the linker is a flexible linker.

3. The vaccine according to any of claims 1 and 2, wherein the linker is non-immunogenic.

4. The vaccine according to any of the preceding claims, wherein the linker is identical in all antigenic subunits.

5. The vaccine according to any of the preceding claims, wherein the linker is a Serine-Glycine linker.

6. The vaccine according to any of the preceding claims, wherein the length of the linker is from 4 to 20 amino acids.

7. The vaccine according to any of the preceding claims, wherein the length of the linker is from 10 to 15 amino acids.

8. The vaccine according to any of the preceding claims, wherein the length of the linker is from 10 amino acids.

9. The vaccine according to any of the preceding claims, comprising one copy of each cancer neoepitope.

10. The vaccine according to any of the preceding claims, comprising at least two copies of at least one cancer neoepitope.

11. The vaccine according to any of the preceding claims, wherein the cancer neoepitope sequence has a length of from 7 to 30 amino acids.

12. The vaccine according to any of the preceding claims, wherein the cancer neoepitope sequence has a length of from 7 to 10 amino acids.

13. The vaccine according to any of claims 1 to 10, wherein the cancer neoepitope sequence has a length of from 13 to 30 amino acids.

14. The vaccine according to any of the preceding claims, wherein each cancer neoepitope sequence has identical length.

15. The vaccine according to any of the preceding embodiments, wherein the cancer neoepitope is positioned essentially in the middle of the cancer neoepitope sequence.

16. The vaccine according to any of the preceding claims, wherein the cancer neoepitope sequence is a subsequence of a cancer neoantigen.

17. The vaccine according to any of the preceding claims, wherein the most hydrophobic antigenic subunit(s) is/are substantially the middle of the antigenic unit and the most hydrophilic antigenic subunit(s) is/are at the ends of the antigenic unit.

18. The vaccine according to any of the preceding claims, wherein the length of the antigenic unit is from about 100 amino acids to about a 1000 amino acids.

19. The vaccine according to any of the preceding claims, wherein n is an integer between 3 and 30.

20. The vaccine according to any of the preceding claims, wherein n is an integer between 10 and 20.

21. A polynucleotide comprising the nucleotide sequence as defined in any of claims 1-20.

22. A polypeptide encoded by the polynucleotide as defined in claim 21.

23. Use of a vaccine as defined in any of claims 1 to 20 for treatment of cancer.

24. A vector comprising the nucleotide sequence as defined in any of the claims 1 to 20.

25. A host cell comprising a nucleotide sequence encoding the antigenic unit as defined in any of the claims 1 to 20 or comprising the vector as defined in claim 23.

26. A method for preparing a vaccine comprising an immunologically effective amount of the polypeptide according to claim 21, the method comprising
e. transfecting a polynucleotide as defined in claim 21 into a cell population;
f. culturing the cell population;
g. collecting and purifying the polypeptide expressed from the cell population
h. mixing the polypeptide obtained under step c) with a pharmaceutically acceptable carrier thereby obtaining the vaccine.

27. A method for preparing a vaccine comprising an immunologically effective amount of the polynucleotide as defined in claim 21, said method comprising
a. preparing the polynucleotide as defined in claim 21;
b. mixing the polynucleotide obtained under step a) with a pharmaceutically acceptable carrier, thereby obtaining the vaccine.

28. The method according to claim 27, further comprising the steps of:
sequencing the exome of a tumour;
identifying tumor neoantigens comprising neoepitopes from said tumor;
selecting neoepitopes based on antigenicity,
prior to the step of preparing the polynucleotide.

29. A method of treating cancer in a patient, the method comprising administering to the patient in need thereof, the vaccine as defined in any of the claims 1 to 20.

30. The method according to claim 29, wherein the vaccine is administered intradermally or intramuscular.

31. The method according to any of claims 29 to 30, wherein the nucleotide sequence is DNA.

32. The method according to any of claims 29 to 30, wherein the nucleotide sequence is RNA.

33. The method according to any of claims 29 to 32, wherein administration is carried out with a jet injector.

34. The method according to any of claims 29 to 33, wherein administration is assisted by electroporation.


SEQUENCE LISTING (77 sequences)

SEQ ID NO: 1 - protein, 27 aa, Mus musculus
PSKPSFQEFVDWENVSPELNSTDQPFL

SEQ ID NO: 2 - protein, 27 aa, Mus musculus
REGVELCPGNKYEMRRHGTTHSLVIHD

SEQ ID NO: 3 - protein, 27 aa, Mus musculus
SHCHWNDLAVIPAGVVHNWDFEPRKVS

SEQ ID NO: 4 - protein, 27 aa, Mus musculus
GRGHLLGRLAAIVGKQVLLGRKVVVVR

SEQ ID NO: 5 - protein, 27 aa, Mus musculus
FRRKAFLHWYTGEAMDEMEFTEAESNM

SEQ ID NO: 6 - protein, 27 aa, Mus musculus
VVDRNPQFLDPVLAYLMKGLCEKPLAS

SEQ ID NO: 7 - protein, 27 aa, Mus musculus
SSPDEVALVEGVQSLGFTYLRLKDNYM

SEQ ID NO: 8 - protein, 27 aa, Mus musculus
EFKHIKAFDRTFANNPGPMVVFATPGM

SEQ ID NO: 9 - protein, 27 aa, Mus musculus
STANYNTSHLNNDVWQIFENPVDWKEK

SEQ ID NO: 10 - protein, 27 aa, Mus musculus
DSGSPFPAAVILRDALHMARGLKYLHQ

SEQ ID NO: 11 - protein, 5 aa, Artificial Sequence
  Linker
GGGGS

SEQ ID NO: 12 - protein, 338 aa, Artificial Sequence
  construct
MQVSTAALAVLLCTMALCNQVLSPSKPSFQEFVDWENVSPELNSTDQPFLGGGGSREGVELCPGNKYEMRRHGTTHSLVIHDGGGGSSHCHWNDLAVIPAGVVHNWDFEPRKVSGGGGSGRGHLLGRLAAIVGKQVLLGRKVVVVRGGGGSFRRKAFLHWYTGEAMDEMEFTEAESNMGGGGSVVDRNPQFLDPVLAYLMKGLCEKPLASGGGGSSSPDEVALVEGVQSLGFTYLRLKDNYMGGGGSEFKHIKAFDRTFANNPGPMVVFATPGMGGGGSSTANYNTSHLNNDVWQIFENPVDWKEKGGGGSDSGSPFPAAVILRDALHMARGLKYLHQ

SEQ ID NO: 13 - protein, 114 aa, Artificial Sequence
  Construct
MQVSTAALAVLLCTMALCNQVLSPSKPSFQEFVDWENVSPELNSTDQPFLGGGGSREGVELCPGNKYEMRRHGTTHSLVIHDGGGGSSHCHWNDLAVIPAGVVHNWDFEPRKVS

SEQ ID NO: 14 - protein, 23 aa, Artificial Sequence
  Signal peptide
MQVSTAALAVLLCTMALCNQVLS

SEQ ID NO: 15 - protein, 5 aa, Artificial Sequence
  Linker
GGGSS

SEQ ID NO: 16 - protein, 5 aa, Artificial Sequence
  Linker
GGGSG

SEQ ID NO: 17 - protein, 5 aa, Artificial Sequence
  Linker
GGGGS

SEQ ID NO: 18 - protein, 5 aa, Artificial Sequence
  Linker
LGGGS

SEQ ID NO: 19 - protein, 5 aa, Artificial Sequence
  Linker
GLGGS

SEQ ID NO: 20 - protein, 5 aa, Artificial Sequence
  Linker
GGLGS

SEQ ID NO: 21 - protein, 5 aa, Artificial Sequence
  Linker
GGGLS

SEQ ID NO: 22 - protein, 5 aa, Artificial Sequence
  Linker
GGGGL

SEQ ID NO: 23 - protein, 5 aa, Artificial Sequence
  Linker
LGGSG

SEQ ID NO: 24 - protein, 5 aa, Artificial Sequence
  Linker
GLGSG

SEQ ID NO: 25 - protein, 5 aa, Artificial Sequence
  Linker
GGLSG

SEQ ID NO: 26 - protein, 5 aa, Artificial Sequence
  Linker
GGGLG

SEQ ID NO: 27 - protein, 5 aa, Artificial Sequence
  Linker
GGGSL

SEQ ID NO: 28 - protein, 5 aa, Artificial Sequence
  Linker
LGGSS

SEQ ID NO: 29 - protein, 5 aa, Artificial Sequence
  Linker
GLGSS

SEQ ID NO: 30 - protein, 5 aa, Artificial Sequence
  Linker
GGLSS

SEQ ID NO: 31 - protein, 5 aa, Artificial Sequence
  Linker
GGGLS

SEQ ID NO: 32 - protein, 5 aa, Artificial Sequence
  Linker
GGGSL

SEQ ID NO: 33 - protein, 5 aa, Artificial Sequence
  Linker
LGLGS

SEQ ID NO: 34 - protein, 5 aa, Artificial Sequence
  Linker
GLGLS

SEQ ID NO: 35 - protein, 5 aa, Artificial Sequence
  Linker
GLLGS

SEQ ID NO: 36 - protein, 5 aa, Artificial Sequence
  Linker
LGGLS

SEQ ID NO: 37 - protein, 5 aa, Artificial Sequence
  Linker
GLGGL

SEQ ID NO: 38 - protein, 5 aa, Artificial Sequence
  Linker
LGLSG

SEQ ID NO: 39 - protein, 5 aa, Artificial Sequence
  Linker
GLLSG

SEQ ID NO: 40 - protein, 5 aa, Artificial Sequence
  Linker
GGLSL

SEQ ID NO: 41 - protein, 5 aa, Artificial Sequence
  Linker
GGLLG

SEQ ID NO: 42 - protein, 5 aa, Artificial Sequence
  Linker
GLGSL

SEQ ID NO: 43 - protein, 5 aa, Artificial Sequence
  Linker
LGLSS

SEQ ID NO: 44 - protein, 5 aa, Artificial Sequence
  Linker
GLGLS

SEQ ID NO: 45 - protein, 5 aa, Artificial Sequence
  Linker
GGLLS

SEQ ID NO: 46 - protein, 5 aa, Artificial Sequence
  Linker
GLGSL

SEQ ID NO: 47 - protein, 5 aa, Artificial Sequence
  Linker
GLGSL

SEQ ID NO: 48 - protein, 10 aa, Artificial Sequence
  Linker
LGGGSGGGGS

SEQ ID NO: 49 - protein, 10 aa, Artificial Sequence
  Linker
GLGGSGGGGS

SEQ ID NO: 50 - protein, 10 aa, Artificial Sequence
  Linker
GGLGSGGGGS

SEQ ID NO: 51 - protein, 10 aa, Artificial Sequence
  Linker
GGGLSGGGGS

SEQ ID NO: 52 - protein, 10 aa, Artificial Sequence
  Linker
GGGGLGGGGS

SEQ ID NO: 53 - protein, 10 aa, Artificial Sequence
  Linker
LGGSGGGGSG

SEQ ID NO: 54 - protein, 10 aa, Artificial Sequence
  Linker
GLGSGGGGSG

SEQ ID NO: 55 - protein, 10 aa, Artificial Sequence
  Linker
GGLSGGGGSG

SEQ ID NO: 56 - protein, 10 aa, Artificial Sequence
  Linker
GGGLGGGGSG

SEQ ID NO: 57 - protein, 10 aa, Artificial Sequence
  Linker
GGGSLGGGSG

SEQ ID NO: 58 - protein, 10 aa, Artificial Sequence
  Linker
GGGSLGGGSG

SEQ ID NO: 59 - protein, 10 aa, Artificial Sequence
  Linker
GLGSSGGGSS

SEQ ID NO: 60 - protein, 10 aa, Artificial Sequence
  Linker
GGLSSGGGSS

SEQ ID NO: 61 - protein, 10 aa, Artificial Sequence
  Linker
GGGLSGGGSS

SEQ ID NO: 62 - protein, 10 aa, Artificial Sequence
  Linker
GGGSLGGGSS

SEQ ID NO: 63 - protein, 10 aa, Artificial Sequence
  Linker
LGGGSLGGGS

SEQ ID NO: 64 - protein, 10 aa, Artificial Sequence
  Linker
GLGGSGLGGS

SEQ ID NO: 65 - protein, 10 aa, Artificial Sequence
  Linker
GGLGSGGLGS

SEQ ID NO: 66 - protein, 10 aa, Artificial Sequence
  Linker
GGGLSGGGLS

SEQ ID NO: 67 - protein, 10 aa, Artificial Sequence
  Linker
GGGGLGGGGL

SEQ ID NO: 68 - protein, 10 aa, Artificial Sequence
  Linker
LGGSGLGGSG

SEQ ID NO: 69 - protein, 10 aa, Artificial Sequence
  Linker
GLGSGGLGSG

SEQ ID NO: 70 - protein, 10 aa, Artificial Sequence
  Linker
GGLSGGGLSG

SEQ ID NO: 71 - protein, 10 aa, Artificial Sequence
  Linker
GGGLGGGGLG

SEQ ID NO: 72 - protein, 10 aa, Artificial Sequence
  Linker
GGGSLGGGSL

SEQ ID NO: 73 - protein, 10 aa, Artificial Sequence
  Linker
LGGSSLGGSS

SEQ ID NO: 74 - protein, 10 aa, Artificial Sequence
  Linker
GLGSSGLGSS

SEQ ID NO: 75 - protein, 10 aa, Artificial Sequence
  Linker
GGLSSGGLSS

SEQ ID NO: 76 - protein, 10 aa, Artificial Sequence
  Linker
GGGLSGGGLS

SEQ ID NO: 77 - protein, 10 aa, Artificial Sequence
  Linker
GGGSLGGGSL